(19) United States
(12) Patent Application Publication
(10) Pub. No.: US 2008/0317769 A1
(43) Pub. Date: Dec. 25, 2008

(54) Vaccine Composition Comprising Alpha-Galactosylceramide as an Adjuvant For Intranasal Administration

(76) Inventors: Chang-Yuil Kang, Seoul (KR); Sung-Youl Ko, Kyonggi-do (KR)

Correspondence Address: KLARQUIST SPARKMAN, LLP, 121 SW SALMON STREET, SUITE 1600, PORTLAND, OR, 97204, US

(21) Appl. No.: 11/995,505

(22) PCT Filed: Apr. 3, 2006
(86) PCT No.: PCT/KR06/01226

§ 371 Date: Apr. 4, 2008

(30) Foreign Application Priority Data
Jul. 13, 2005 (KR) 10-2005-0063431

(51) Int. Cl. A61K 39/00; A61P 43/00

(52) U.S. Cl. 424/184.1

(57) ABSTRACT

The present invention related to a vaccine composition comprising alpha-galactosylceramide (αGalCer) as an adjuvant for the intranasal administration. The present inventors administered αGalCer together with a tumor cell antigen or a virus antigen to the nasal cavity of a mouse and then confirmed that the αGalCer effectively induced not only humoral immunity but also cell-mediated immunity. Thus, the αGalCer can be effectively used as an adjuvant for a vaccine by the intranasal administration for the prevention and treatment of virus infection and cancer.

### TECHNICAL FIELD

The present invention relates to a vaccine composition comprising alpha-galactosylceramide (α-GalCer) as an adjuvant for the intranasal administration.

### BACKGROUND ART

As of today, new vaccines for the treatment of various neoplastic and infectious diseases have been developed. Unlike the conventional vaccines using live attenuated or non-replicating inactivated pathogens, current vaccines are composed of synthetic, recombinant or purified subunit antigens.

In spite of a variety of studies to treat cancer by immunotherapy using human immune system, appropriate antibody immune response has not been induced or tumor specific cytotoxic T-cells have not been activated properly since human cancer cells are not antigen presenting cells.

Vaccines have also been used as a major tool to reduce the chances of hospitalization and a death rate of a patient with viral infection, such as influenza virus infection. But, the RNA virus such as influenza virus is characterized by continuous antigenic variation, making the development of a vaccine for the virus difficult. Nevertheless, there have been efforts to develop proper vaccines for viruses, such as influenza virus, SARS and so on, because they cause world threatening infectious diseases.

The major invasion routes of an antigen are oral cavity, nasal cavity, larynx, small intestine, large intestine, genitalia and anus, and the mucosal system is the primary defense line for a pathogenic antigen, forming the mucosal immune system, which is one of the two major immune systems (the other is systemic immune system). Therefore, most studies to develop a vaccine have been focused on the development of a vaccine composition that is able to induce both mucosal and systemic immune responses (Czerkinsky et al., Immunol. Rev., 170: 197, 1999; Belyakov et al., Proc, Natl. Acad. Sci. U.S.A., 95: 1709, 1998; Berzofsky et al., Nat. Rev. Immunol., 1: 209, 2001; Kozlofsky et al., Curr. Mol. Med., 3: 217, 2003).

A vaccine can be developed in various formulations. Considering compliance of a patient, dosage, easiness of administration and occurrence rate of side effects, the most ideal formulation is an intranasal vaccine.

The injection of a vaccine with needle reduces the compliance of a patient by causing pains on the injection area where might involve a risk of infection. In the meantime, the mucosal vaccination, for example a nasal vaccination, avoids the injection with a needle. Thus, the mucosal vaccination is much easier and more convenient way than the conventional injection vaccination. Moreover, the intranasal vaccination has several advantages comparing with the conventional oral vaccination in that intranasal administration avoids hepatic first pass effect and degradation of administrated antigen in the gastrointestinal tract, which brings high bioavailability, cost-reduction and low side effect occurrence rate owing to the minimum dosage (Remeo et al., Adv. Drug Deliv. Rev., 29: 89, 1998).

The mucosal vaccine comprising antigens alone induces immune tolerance rather than immune response, so co-administration with an adjuvant is essential (Yuki et al., Rev. Med. Virol., 13: 293, 2003). But, a clinically acceptable adjuvant for inducing mucosal immunity has not been reported yet even though an adjuvant inducing mucosal immunization is in urgent need.

The ‘adjuvant’ means any compound that promotes or amplifies a specific stage of immune response so as to enhance the immune response at last. The administration of an adjuvant alone does not affect immunity but the co-administration with a vaccine antigen can increase and keep up the immune response against the antigen. An adjuvant is typically exemplified by oil emulsion (Freund's adjuvant), saponin, aluminum or calcium salts (alum), non-ionic block polymer surfactants, lipopolysaccharides, mycobacteria and tetanus toxoid.

αgalactosylceramide (α-GalCer) is a glycolipid originated from marine sponge, Agelas mauritianus, which acts as a ligand for Vα14+ T cell receptor (TCR) of NKT (Natural Killer T) cell and is presented by CD1d of antigen presenting cell (APC) (Kawano et al., Science, 278: 1626, 1997). The activation of NKT cells leads to the production of IFN-γ and IL-4, providing the chances of regulation of immune response for a specific disease or infection (Chen et al., J. Immunol., 159: 2240, 1997; Wilson et al., Proc. Natl. Acad. Sci. U.S.A., 100: 10913, 2003).

In previous studies, the role and effect of αGalCer as an adjuvant for the systemic vaccination were examined. As a result, αGalCer was confirmed to act as an effective adjuvant for the treatment of infections (Gonzalez-Aseguinolaza et al., Proc. Natl. Acad. Sci. U.S.A., 97: 8461, 2000; Gonzalez-Aseguinoalza et al., J. Exp. Med., 195: 615, 2002), auto-immune diseases (Laloux et al., J. Immunol., 166: 3749, 2001: Teige et al., J. Immunol., 172: 186, 2004) and cancers (Hermans et al., J. Immunol., 171: 5140, 2003; Fujii et al., J. Exp. Med., 199: 1607, 2003; Hayakawa et al., Proc. Natl. Acad. Sci. U.S.A., 100: 9464, 2003).

According to WO 2003/009812, when αGalCer was administered as an adjuvant by intraperitoneal injection, intramuscular injection and intravenous injection, it increased antigen specific Th1-type response, particularly CD8+ T cell response. Korean Patent Publication No. 2003-0017733 also describes that when tumor lysate and αGalCer are co-injected into the abdominal cavity, NKT cells are stimulated to increase the expression of a cofactor for T cell activation, resulting in the inhibition of tumor cell growth.

However, the above documents only proved that αGalCer induces cell mediated immune response by the systemic administration as an adjuvant and do not mention the functions of αGalCer as an adjuvant for the nasal vaccination.

Since immunological microenvironments and dynamics of immune cells in different lymphoid organs differ, it isn't accepted that a certain adjuvant inducing immune responses via systemic route can also be used as a nasal vaccine adjuvant or vice versa in the respects of immunology. Particularly, in the aspects of humoral immune response and cell mediated immune response, a nasal vaccine and an intramuscular or a subcutaneous vaccine might induce different immune responses. Thus, thorough examination is required to verify whether an adjuvant for an intramuscular vaccine can be used as an adjuvant for a nasal vaccine. For example, alum is the only vaccine adjuvant for clinical use that is administered by intramuscular injection, but cannot be used as an adjuvant for a nasal vaccine. Cholera toxin is a promising candidate for a nasal vaccine adjuvant but not a target of the study on an intramuscular vaccine adjuvant. The most important immune response against pathogens invading through mucosa is the generation of secretory IgA that is only induced by mucosal vaccination. Besides, mucosal vaccination can induce both mucosal immune response and systemic immune response, so that it induces immune responses against pathogens not only through mucosa but also through other routes. Therefore, an adjuvant for intramuscular vaccine or a vaccine for systemic administration cannot be used as an adjuvant for a vaccine for the intranasal administration. To use an adjuvant for different administration methods, it has to be verified experimentally and clinically (Infectious Disease Review 3:2, 2001; Nature Immunology 6: 507, 2005; Reviews in Medical Virology, 2003, 13:293-310; Nature Reviews Immunology, 1: 20, 2001).

The present inventors co-administered a tumor-associated antigen or a virus antigen and αGalCer to the nasal cavity of a mouse and confirmed that the co-treated αGalCer induced not only humoral immune response but also cell mediated immune response against the tumor-associated or the virus antigen. And the present inventors completed this invention by further confirming that αGalCer can be used as an adjuvant for a nasal vaccine composition.

### DISCLOSURE

### Technical Problem

It is an object of the present invention to provide a composition for the prevention and treatment of virus infection and cancer comprising αGalCer as an adjuvant for a nasal vaccine composition, which has been confirmed by the inventors to induce both humoral immune response and cell mediated immune response against a tumor-associated antigen or a virus antigen administered in the nasal cavity of mice.

### Technical Solution

The present invention provides a nasal vaccine composition containing an antigen and an effective dose of alpha-galactosylceramide as an adjuvant.

The present invention also provides a method to enhance systemic immune response and mucosal immune response, simultaneously, against an antigen co-administered with alpha-galactosylceramide to the nasal cavity.

The present invention further provides a method to enhance both Th1 and Th2 immune responses by the intranasal administration of the vaccine composition.

The present invention also provides a method to enhance secretory IgA production in mucosal compartment and IgG production in systemic compartment by the intranasal administration of the vaccine composition.

The present invention also provides a vaccine adjuvant comprising alpha-galactosylceramide for intranasal administration.

Hereinafter, the present invention is described in detail.

α-galactosylceramide (αGalCer) is a glycolipid originated from marine sponge, which acts as a ligand for Vα14+ T cell receptor (TCR) of NKT (Natural Killer T) cell and is presented by CD1d molecule of antigen presenting cell (APC) (Kawano et al., Science, 278: 1626, 1997). The activation of NKT cells leads to the production of IFN-γ and IL-4, providing the chances of regulation of immune response for a specific disease or infection (Chen et al., J. Immunol., 159: 2240, 1997; Wilson et al., Proc. Natl. Acad. Sci. U.S.A., 100: 10913, 2003). According to some of the previous reports, activated NKT cells can induce Th2 immune response (Yoshmoto et al., Science, 270: 1845, 1995; Singh et al., J. Immunol. 163: 2373, 1999; Laloux et al., J. Immunol., 166: 3749). But, others say that activated NKT cells induce Th1 immune response (Hermans et al., j. Immunol., 171: 5140, 2003; Stober et al., J. Immunol., 170: 2540, 2003). According to recent reports, the co-treatment of αGalCer and OVA induces complete maturation of dendritic cells (DC) and thereby induces antigen-specific Th1 CD4+T cells and CTL having resistance against OVA expressing tumors (Fujii et al., J. Exp. Med., 198: 267, 2003; Fujii et al. J. Exp. Med., 199: 1607, 2004). Additionally, the present inventors successfully inhibited oral tolerance induced by both high and low amount of an antigen in vitro by inducing full maturation of DC and T cell differentiation in mesenteric lymph node after the systemic administration of αGalCer and oral administration of OVA (Chung et al., Eur. J. Immunol., 34: 2471, 2004). The result indicates that αGalCer can be used as an effective adjuvant for various mucosal vaccines and induce Th1 and CTL or Th2 immune responses.

The present inventors further confirmed that the intranasal administration of OVA together with αGalCer induced OVA-specific mucosal S-IgA and systemic IgG antibody response, Th1 and Th2 cytokine responses and very strong CTL response as well in both C57BL/6 and Balb/c mice.

To investigate the activity of αGalCer as an adjuvant in mucosa, required amount of αGalCer and 100 μg of OVA or 100 μg of OVA alone was diluted with PBS, making 20 μl solution (10 μl/nostril), which was administered to C57BL/6 mice or Balb/c mice (Charles River Laboratories, Orient Co., Ltd., Korea) at 6-8 weeks three times at one-week intervals.

αGalCer was provided from Dr. Snaghee Kim (Seoul National University, Korea), which was prepared by linking phytosphingosine to hexacosanoic acid and then performing protection/deprotection and galactosylation according to the conventional art (Takikawa et al., Tetrahedron, 54: 3141, 1998). αGalCer was dissolved in PBS containing 0.5% tween 20. PBS containing 0.5% tween 20 was used as a vehicle for every experiment herein.

From the investigation on humoral immune response against OVA in C57BL/6 mice, it was confirmed that αGalCer increased the level of antigen-specific mucosal S-IgA (Secretory IgA) (see FIG. 1) and the levels of OVA-specific Th2 type IgG1 and Th1 type IgG2a as well, indirectly suggesting that αGalCer induces both Th1 and Th2 immune responses (see FIG. 2 and FIG. 3). The levels of Th1 type cytokine IFN-γ and Th2 type cytokine IL-4 in spleen and CLN were significantly increased by αGalCer, directly indicating that αGalCer induces both Th1 and Th2 immune responses (see FIG. 4).

The above results indicate that αGalCer is a powerful mucosal vaccine adjuvant that is able to induce both antigen-specific mucosal S-IgA (Secretory IgA) and systemic IgG antibody response and induce both Th1 and Th2 immune responses in C57BL/6 mice.

It has been well established that αGalCer induces CTL response when it is administered intravenously or orally (Fujii et al, J. Exp. Med., 198: 267, 2003: Silk et al., J. Clin. Invest., 114: 1800, 2004). Thus, it was further investigated whether αGalCer could induce CTL response in C57BL/6 mice when it is administered to the nasal cavity together with OVA. As a result, all the groups treated with αGalCer showed dose-dependent lytic activity and cytotoxic activity in mucosal (CLN) and systemic (spleen and MLN) compartments (see FIG. 5 and FIG. 6). The above results indicate that αGalCer is a powerful nasal vaccine adjuvant that is able to induce CTL in both mucosal and systemic immune systems. The result of the investigation on αGalCer activity in Balb/c mice was consistent with the above results, suggesting that the effect of αGalCer is not limited to C57BL/6 mice (see FIG. 7-FIG. 11).

αGalCer has a nasal vaccine adjuvant activity that is able to induce an antiviral immune response particularly against influenza virus A/PR/8/34 infection. To investigate how much the mucosa is protected by αGalCer against the virus infection, Balb/c mice were immunized with αGalCer and PR8 HA antigen by the intranasal administration three times at one-week intervals. Two weeks after the final immunization, 20 LD₅₀ of influenza virus was challeged through nasal route. Three days later, PR8 HA-specific antibody response was measured in nasal wash, lung wash and blood serum. As a result, high level of PR8 HA-specific IgA antibody was detected in nasal wash, lung wash and blood serum of all αGalCer-treated groups (see FIG. 12) and high level of PR8 HA-specific IgG antibody was also detected in the blood serum of all mice coimmunized with αGalCer (see FIG. 13). Therefore, it was confirmed that αGalCer is a powerful nasal vaccine adjuvant that induces not only systemic IgG but also mucosal S-IgA against a virus antigen. Pathogenesis was much more severe in mice immunized with antigen alone than in those coimmunized with antigen and αGalCer (see FIG. 14). All the mice treated with vehicle alone died within 10 days and 57% of the mice treated with PR8 HA alone died within 14 days after virus infection. On the contrary, the mice coimmunized with αGalCer and PR8 HA by intranasal route did not show any significant decrease in survival rate and weight loss, and rapid rate of weight loss recovery (see FIG. 14). Therefore, αGalCer was confirmed to be a powerful nasal vaccine adjuvant that is able to induce strong defense mechanism against virus infection and mucosal S-IgA antibody as well as systemic IgG antibody.

The immune responses induced by αGalCer nasal vaccine adjuvant was further investigated by immunizing a Balb/c mouse with 0.125 μg of αGalCer and replication-defective adenovirus harboring β-galactosidase gene (Ad-LacZ) (Viromed, Korea) by intranasal route. As a result, αGalCer effectively induced cell mediated and humoral immune responses against the replication-defective adenovirus harboring β-galactosidase gene (see FIG. 15-FIG. 17).

It was further confirmed that αGalCer has a nasal vaccine adjuvant activity to induce anticancer immune response against EG7 tumor. C57BL/6 mice were immunized with OVA together with αGalCer by intranasal administration three times at one-week intervals. Two weeks after the final immunization, 3×10⁶ EG7 tumor cells were subcutaneously inoculated in the left flank of the immunized mice. 14 days after the inoculation, the mice were sacrificed and palpable tumors were excised out and the weights were measured. As a result, tumor formations were completely inhibited in the mice coimmunized with 0.5 μg and 2.0 μg of αGalCer and OVA by intranasal route (see FIG. 18). These results indicate that αGalCer can be used as a potent nasal vaccine adjuvant to induce anticancer immune response.

To investigate whether the immune responses induced by α-GalCer nasal vaccine adjuvant are mediated by CD1d molecule, CD1d−/− C57BL/6 mice, in which CD1d molecule is deficient and thereby NKT cells are deficient, were intranasally immunized with OVA alone or together with x-GalCer three times at one-week intervals. One week later, systemic IgG response in serum and in vivo CTL activity were investigated in both wild type and the CD1d−/− C57BL/6 mouse. As a result, systemic IgG antibody response in CD1d−/− mouse was significantly inhibited (see FIG. 19) and CTL lytic activity was also inhibited in the draining lymph node and the systemic lymphoid organs of the CD1d−/− mouse (see FIG. 20). The above results indicate that the immune responses induced by α-GalCer nasal vaccine adjuvant are mediated exclusively by CD1d molecule.

The intranasal administration of αGalCer induces the activation of naïve T cells and thereby differentiates those cells into effector cells. To re-confirm the effect of αGalCer on the naive T cell activation, CFSE-labeled OT1 cells were adoptively transferred to syngenic mice. On the next day of the adoptive transfer, OVA alone or OVA together with 2.0 μg of αGalCer was intranasally administered to the mice. 48 hours later, CD25 expression in CLN was investigated. As a result, the level of CD25 expressing OT1 cells was higher in the mice co-treated with OVA and αGalCer than in those treated OVA alone, which means αGalCer nasal adjuvant induces the activation of naive T cells (see FIG. 21). To confirm whether the activated T cells were successfully differentiated into highly functional CTL, those cells were further stimulated with OVA₂₅₇₋₂₆₄ peptide for 6 hours and then intracellular IL-2 and IFN-γ levels were measured. As a result, the levels of IL-2 and IFN-γ produced by OT1 cells were higher in the mice immunized with OVA together with αGalCer by intranasal route than in those treated with OVA alone (see FIG. 22). The results indicate that the intranasally administered αGalCer induces the activation of naïve T cells and triggers the activated T cells to differentiate into effector T cell.

αGalCer induced authentic and powerful immune response against influenza infection even in the case of immunization with killed PR8 virus as an antigen. Particularly, Balb/c mice were immunized with killed PR8 virus and αGalCer by intranasal route twice at two-week intervals. As a result, αGalCer nasal vaccine adjuvant increased the level of IgG in serum (see FIG. 23) and that of S-IgA in mucosal compartment (see FIG. 24). αGalCer nasal vaccine adjuvant also significantly increased the proliferation of immune cells (see FIG. 25) and the productions of IFN-γ and IL-4 (see FIG. 26). The cytotoxic T cells activated by αGalCer nasal vaccine adjuvant were proved to have strong lytic activity (see FIG. 27) and protective immunity (see FIG. 28). The above results indicate that αGalCer, when it is co-treated with even a killed virus antigen via intranasal route, induces powerful humoral immune response and cell mediated immune response as well as strong and authentic protective immune response against live virus infection.

The above results also suggest that αGalCer can be used as an effective nasal vaccine adjuvant to induce anti-infection and anticancer immune response.

Thus, the present invention provides a vaccine composition comprising the effective dose of α-galactosylceramide adjuvant and an antigen.

Herein the term “effective dose of adjuvant” indicates the amount of αGalCer that is able to promote immune response against an antigen administered by intranasal route, which is also well understood by those in the art. More precisely, the effective dose of adjuvant means the amount that is able to increase the level of S-IgA more than 5%, more preferably 25% and most preferably more than 50% in the nasal wash from mice coimmunized with an antigen and α-GalCer, compared with that with an antigen alone.

Therefore, it is preferred for the composition of the invention to contain α-galactosylceramide less than 0.5 w/v %.

“Antigen” means any substance that is able to induce immune response by being recognized by a host immune system when it invades into a host (for example, protein, peptide, cancer cell, glycoprotein, glycolipid, live virus, killed virus, DNA, etc.).

An antigen can be provided either as a purified form or a non-purified form, but a purified form is preferred.

The present invention can be applied to various antigens including protein, recombinant protein, peptide, polysaccharide, glycoprotein, glycolipid and DNA (polynucleotide) of a pathogen, cancer cell, live virus and killed virus.

The following list of antigens is provided as a reference for exemplary embodiments of the invention but not limited thereto: influenza virus antigen (haemagglutinin and neuraminidase antigens), Bordetella pertussis antigen (pertussis toxin, filamentous haemagglutinin, pertactin), human papilloma virus (HPV) antigen, Helicobacter pylori antigen (capsula polysaccharides of serogrup A, B, C, Y and W-135), tetanus toxoid, diphtheria antigen (diphtheria toxoid), pneumococcal antigen (Streptococcus pnemoniae type 3 capsular polysaccharide), tuberculosis antigen, human immunodeficiency virus (HIV) antigen (GP-120, GP-160), cholera antigen (cholera toxin B subunit), staphylococcal antigen (staphylococcal enterotoxin B), shigella antigen (shigella polysaccharides), vesicular stomatitis virus antigen (vesicular stomatitis virus glycoprotein), cytomegalovirustigen (CMV) antigen, hepatitis antigen (hepatitis A (HAV), B (HBV), C(HCV), D (HDV) and G (HGV) antigen), respiratory synctytial virus (RSV) antigen, herpes simplex antigen or their combination (Ex, diphtheria, pertussis and tetanus, DPT).

The nasal vaccine composition of the present invention can be formulated as a liquid or a powder type composition, particularly, aerosols, drops, inhaler or insufflation according to the administration methods, and powders or microspheres are preferred.

A composition for nasal drops can include one or more acceptable excipients such as antiseptics, viscosity regulators, osmotic regulators and buffers.

The administration amount of a vaccine is determined as the amount that is able to induce immune response effectively. For example, the administration frequency of a vaccine to human is once to several times a day and the dosage is 1-250 μg and preferably 2-50 μg.

α-galactosylceramide seems not to induce toxicity in rodents and apes (Nakata et al., Cancer Res., 58: 1202-1207, 1998). And, no side effects have been report in a mouse treated with 2200 μg/Kg of αGalCer and αGalCer was proved to be a safe substance that does not cause dose-limiting toxicity (50-4800 μg/m²) and to have resistance during dose escalation study (Giaccone et al., Clin. Cancer Res., 8: 3702, 2002).

The present invention also provides a method to enhance immune responses against an antigen administered with αGalCer through intranasal route.

The concurrent administration of the above mentioned antigen together with αGalCer into the nasal cavity is preferably performed by the dispensing device and the aerosol delivery system is more preferably used.

The present invention further provides a method to enhance Th1 and Th2 immune response by the concurrent administration of the antigen together with αGalCer into the nasal cavity.

The present invention also provides a method to enhance IgA mucosal immune response and IgG systemic immune response by the concurrent administration of the antigen together with αGalCer into the nasal cavity.

The present invention provides a nasal vaccine composition containing α-GalCer as a potent nasal vaccine adjuvant.

### DESCRIPTION OF DRAWINGS

The application of the preferred embodiments of the present invention is best understood with reference to the accompanying drawings, wherein:

FIG. 1-FIG. 4 illustrate that the co-administration of OVA and αGalCer induced OVA-specific S-IgA and systemic IgG responses and Th1 and Th2 cytokine secretions in C57BL/6 mice.

FIG. 1 is a set of graphs showing the OVA-specific S-IgA titers in the nasal wash (NW) and the lung wash (LW) of mice one week after the final immunization with OVA alone or together with αGalCer by intranasal route three times at one-week intervals.

FIG. 2 is a graph showing the OVA-specific systemic IgG titer in the serum, and

FIG. 3 is a graph showing the OVA-specific IgG isotype titers in the serum.

FIG. 4 is a set of graphs showing the levels of IFN-γ and IL-4 production in the culture supernatant obtained after the culture of OVA and single cells from spleen and cervical lymph node (CLN) for four days, which were examined by sandwich ELISA.

FIG. 5 and FIG. 6 illustrate that αGalCer induces a strong CTL response in vivo in C57BL/6 mice.

FIG. 5 is a set of graphs illustrating the specific lysis of spleen cells analyzed by FACS. Particularly, equal numbers of OVA₂₅₇₋₂₆₄ peptide pulsed CFSE^{high} spleen cells (target cells) and unpulsed CFSE^{low} spleen cells (control cells) from naïve C57BL/6 mice were intravenously injected to immunized mice. 24 hours later, the mice were sacrificed and the proportions of target cells were measured in spleen, MLN and CLN.

FIG. 6 is a set of graphs presenting the CTL activities measured in FIG. 5 as a percentage.

FIG. 7-FIG. 11 illustrate that the co-administration of OVA and αGalCer by intranasal route induced OVA-specific antibody response, Th1 and Th2 cytokine secretions and CTL activity in Balb/c mice.

FIG. 7 is a set of graphs showing OVA-specific S-IgA titers in the nasal wash (NW) and the lung wash (LW) one week after the final immunization.

FIG. 8 is a graph showing OVA-specific systemic IgG titer in serum.

FIG. 9 is a graph showing IgG isotype titers in serum.

FIG. 10 is a set of graphs showing the levels of IFN-γ and IL-4 in the culture supernatant obtained after the culture of OVA and single cells from spleen and cervical lymph node (CLN) for four days, which were examined by sandwich ELISA.

FIG. 11 illustrates the production of IFN-γ-producing CD8⁺ T cells (CTL) after the culture of splenocytes and OVA for 4 days and examined by intralcellular cytokine staining (ICS).

FIG. 12-FIG. 14 illustrate the strong protective immune responses induced by α-GalCer nasal vaccine adjuvant against influenza virus A/PR/8/34 infection in Balb/c mice.

FIG. 12 is a set of graphs showing PR8 HA-specific S-IgA titers in the nasal wash (NW), the lung wash (LW) and serum. Particularly, Balb/c mice were immunized with PR8 HA alone or together with αGalCer by intranasal route three times at one-week intervals. 2 weeks later, the mice were infected with 20 LD₅₀ of live influenza virus A/PR/8/34 through intranasal route. Then, PR8 HA-specific S-IgA titers in nasal wash (NW), the lung wash (LW) and serum were measured.

FIG. 13 is a graph showing PR8 HA-specific IgG titer in serum.

FIG. 14 is a set of graphs showing the survival rates and weight loss of mice measured every other day after the virus infection.

FIG. 15-FIG. 17 illustrate that intranasally administered αGalCer induced mucosal S-IgA and systemic IgG responses as well as CTL response in Balb/c mice, establishing the strong immunity against replication-deficient live adenovirus infection.

FIG. 15 is a set of graphs showing β-galactosidase-specific S-IgA titers in the nasal wash (NW) and the lung wash (LW), measured one week after immunization of Balb/c mice with replication-deficient live adenovirus alone or together with αGalCer by intranasal route twice at 2-week intervals.

FIG. 16 is a graph showing β-galactosidase-specific IgG titer in serum.

FIG. 17 is a graph showing the level of IFN-γ-producing CD8+ T cells measured by intracellular cytokine staining after stimulating spleen cells with β-galactosidase.

FIG. 18 is a graph illustrating that the co-administration of OVA and αGalCer through the nasal cavity of a C57BL/6 mouse could induce a strong protection against EG7 tumor. Particularly, after 2 weeks from the final immunization, 3×10⁶ EG7 tumor cells were subcutaneously inoculated in the left flank of the immunized mice. 14 days later, the weight of palpable tumors and occurrence rate of the tumor were investigated.

FIG. 19 and FIG. 20 illustrate that the activity of αGalCer as an adjuvant is mediated by CD1d.

FIG. 19 is a graph showing OVA-specific IgG titers in the serums of wild type and CD1d−/− C57BL/6 (CD1d−/−) mice. Shortly, wildtype and CD1d−/− C57BL/6 mice were immunized with OVA together with α-GalCer three times at one-week intervals. One week after the final immunization, equal numbers of OVA₂₅₇₋₂₆₄ pulsed CFSE^{high} splenocytes (target cell) and unpulsed CFSE^{low} splenocytes (control cell) were adoptively transferred to the immunized mice. One day later, OVA-specific IgG titer in serums were measured by ELISA and showed in FIG. 19, and the proportions of target cells were examined by FACS and showed in FIG. 20.

FIG. 21 and FIG. 22 illustrate that the co-administration of OVA and αGalCer through intranasal route activates naïve CD8+ T cells and thereby induces the differentiation of them into effector T cells.

FIG. 21 is a set of graphs showing the activation of naïve T cells by α-GalCer nasal vaccine adjuvant. CFSE-labeled OT-1 cells were adoptively transferred into syngenic mice. One day later, the mice were intranasally immunized with OVA together with α-GalCer. One day later, lymphoid cells from CLN were analyzed for the surface expression of CD25 by FACS.

FIG. 22 is a set of graphs showing that α-GalCer nasal vaccine adjuvant triggers the activated T cells to differentiate into effector T cells. The lymphoid cells obtained as in FIG. 21 were further examined the production of intracellular IL-2 and IFN-γ after stimulation of the cells with OVA₂₅₇₋₂₆₄ peptide and GolgiPlug (BD Pharmingen) for 6 hours by FACS.

FIG. 23-FIG. 28 illustrate that the immunization with formaline-inactivated PR8 virus together with αGalCer through intranasal route induces humoral immune response, cell mediated immune response and protective immune response. Balb/c mice were immunized with inactivated PR8 virus together with αGalCer by intranasal route twice at two-week intervals. Two weeks after the final immunization, the mice were sacrificed and the nasal wash and the lung wash were obtained. The productions of IgG (FIG. 23) and mucosal S-IgA (FIG. 24) therein were measured.

FIG. 25 shows the proliferation of immune cells in single cells separated from spleen and CLN.

FIG. 26 is a set of graphs showing the productions of Th1 and Th2 cytokines.

FIG. 27 is a graph showing the result of ⁵¹Cr release assay to measure CTL activity.

FIG. 28 is a graph illustrating that the immunized mice were infected with live PR8 virus and then the numbers of the virus in the lung wash were measured by plaque assay to investigate protective immune response.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### OVA-Specific Mucosal S-IgA and Systemic IgG Antibody Responses Induced by the Intranasal Co-Administration of an Antigen and αGalCer to C57BL/6 Mice

Six to eight-weeks-old C57BL/c mice (Charles River Laboratories, Orient Co., Ltd., Korea) were immunized with 100 μg of OVA alone or together with the indicated amounts of αGalCer (0.125, 0.5, 2.0 μg), diluted with PBS and made 20 μl (10 μl/nostril) solution, three times at one-week intervals.

αGalCer was provided from Dr. Sanghee Kim (Seoul National University, Korea), which was prepared by linking phytosphingosine to hexacosanoic acid and then performing protection/deprotection and galactosylation according to the conventional art (Takikawa et al., Tetrahedron, 54: 3141, 1998). αGalCer was dissolved in PBS containing 0.5% tween 20. PBS containing 0.5% tween 20 was used as a vehicle for every experiment herein.

A week after the final immunization, the mice were sacrificed. OVA-specific antibody responses were measured by ELISA. The nasal wash sample was obtained by washing the nasal passage with 100 μl of sterilized PBS (Yamamoto et al., J. Immunol., 161: 4115, 1998), and bronchoalveolar lavage fluid was also obtained by the same manner as described to prepare the lung wash (Chung et al., Immunobiology 206: 408, 2002).

OVA-specific IgG titers in the nasal wash and the lung wash were measured (Chung et al., Immunobiology 206: 408, 2002). To measure IgA, IgG1 and IgG2a titers, two-fold serially diluted samples were used. To determine IgA titer, horseradish-peroxidase-conjugated goat anti-mouse IgA (SIGMA, USA), peroxidase substrate and TMB (SIGMA, USA) were used and 0.5 N—HCL was added thereto to terminate color development. Then, OD₄₅₀ was measured. To determine IgG, IgG1 and IgG2a titers, alkaline phosphatase-conjugated goat anti-mouse IgG, IgG1 and IgG2a (Southern Biotech, USA) and alkaline phosphatase substrate, p-nitrophenyl phosphate (SIGMA), were used.

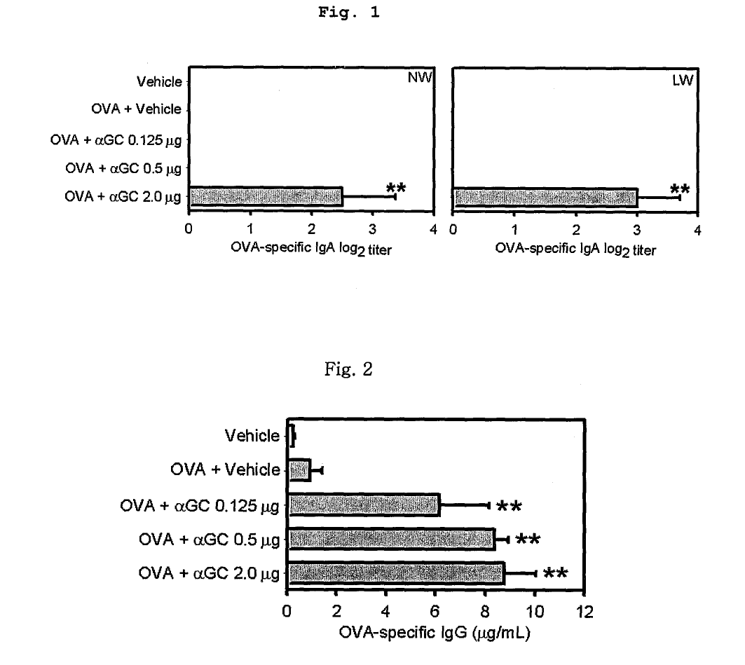

As shown in FIG. 1, OVA-specific IgA responses in the nasal wash and the lung wash were significantly higher in mice coimmunized with 2.0 μg of αGalCer than in those immunized with vehicle alone or OVA alone.

As shown in FIG. 2, higher levels of OVA-specific IgG were detected in serums of mice coimmunized with different concentrations of αGalCer (0.125, 0.5, 2.0 μg) than those immunized with vehicle alone or OVA alone.

To assess the immune bias towards Th1 or Th2 immune responses induced by α-GalCer nasal vaccine adjuvant indirectly, IgG isotypes in serum were determined and the ratios of IgG1 to IgG2a were calculated.

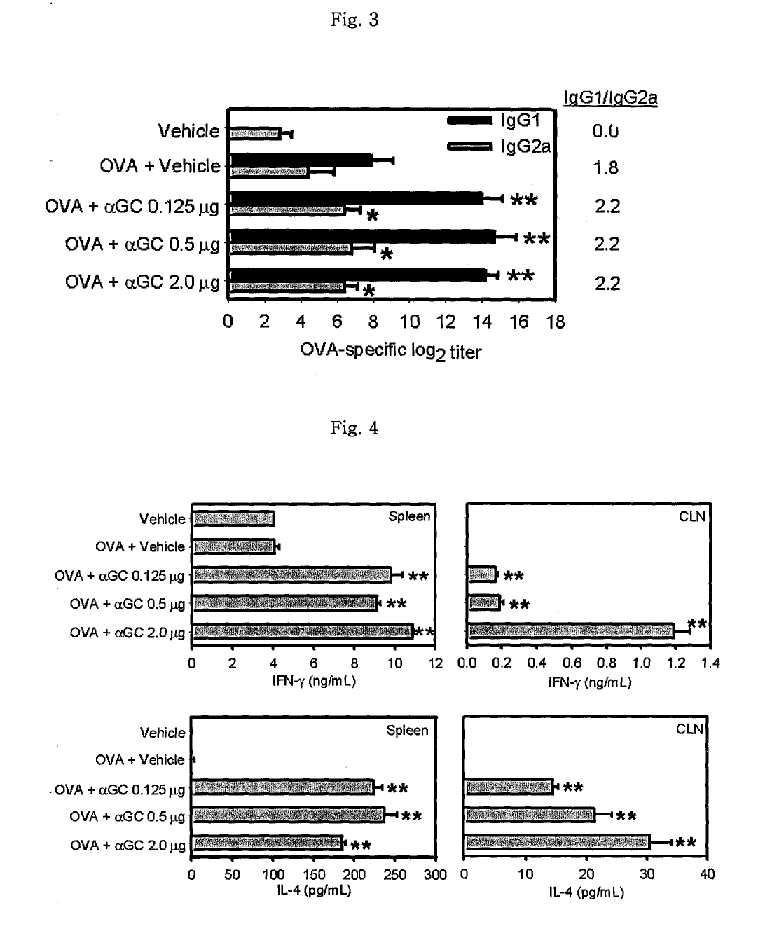

As shown in FIG. 3, the co-administration of αGalCer and OVA resulted in the remarkable increase in the levels of OVA-specific Th2 type IgG1 and Th1 type IgG2a, indicating that αGalCer nasal vaccine adjuvant didn't skew immune responses into Th1 or Th2 immune responses and induced both Th1 and Th2 immune responses.

From the above results, it was confirmed that αGalCer is a strong mucosal adjuvant that is able to induce an antigen-specific mucosal S-IgA (Secretory IgA) and systemic IgG antibody responses and can induce both Th1 and Th2 immune responses in C57BL/6 mice.

### Example 2

### Secretion of Th1 and Th2 Cytokines by the Intranasal Co-Administration of an Antigen and αGalCer to C57BL/6 Mice

It was directly investigated whether α-GalCer nasal vaccine adjuvant skews immune response into Th1 or Th2 immune response. To measure the secretions of cytokines, cells were obtained from spleen and cervical lymph node (CLN) a week after the final immunization. The cells (5×10⁶ cells/μl) were cultured with 500 μg/ml of OVA for 4 days. The secretions of IFN-γ and IL-4 in the culture supernatant were measured by using the mouse IFN-γ and IL-4 OptELA set ELISA kit (BD Pharmigen) according to the manufacturer's instruction.

As shown in FIG. 4, the secretions of IFN-γ and IL-4 in spleen and CLN were significantly increased. High concentration of αGalCer induced IFN-γ secretion more and the secretion of IL-4 in CLN was also increased in the proportion to the concentration of αGalCer.

From the above results, it was confirmed that the intranasal administration of αGalCer induces both Th1 (IFN-γ) and Th2 (IL-4) immune responses in both systemic (spleen) and mucosal (CLN) compartments.

### Example 3

### Strong CTL Response Induced by the Intranasal Co-Administration of an Antigen and αGalCer to C57BL/6 Mice

It has been well-known that the intravenous or oral administration of αGalCer induces CTL response (Fuji et al, J. Exp. Med., 198: 267, 2003: Silk et al., J. Clin. Invest., 114: 1800, 2004). Herein, whether the intranasal administration of αGalCer could induce CTL response was investigated.

Spleen cells were separated from naive C57BL/6 mice, which were pulsed with 1 μM of OVA₂₅₇₋₂₆₄ at 37° C. for 90 minutes. The pulsed cells were labeled with 20 μM of CFSE (Molecular Probes, USA) at 37° C. for 15 minutes, resulting in OVA₂₅₇₋₂₆₄ pulsed CFSE^{high} cells. In the meantime, the unpulsed cells were labeled with 2 μM of CFSE (Molecular Probes, USA) at 37° C. for 15 minutes, resulting in the OVA₂₅₇₋₂₆₄ unpulsed CFSE^{low} cells. The equal numbers of peptide-pulsed CFSE^{high} cells and unpulsed CFSE^{low} cells were mixed, which were intravenously injected to mice at the number of 2×10⁷ cells one week after the final immunization. 24 hours later, specific lysis of peptide-pulsed CFSE^{high} cell was investigated by using FACS in spleen, mesenteric lymph node (MLN) and cervical lymph node (CLN).

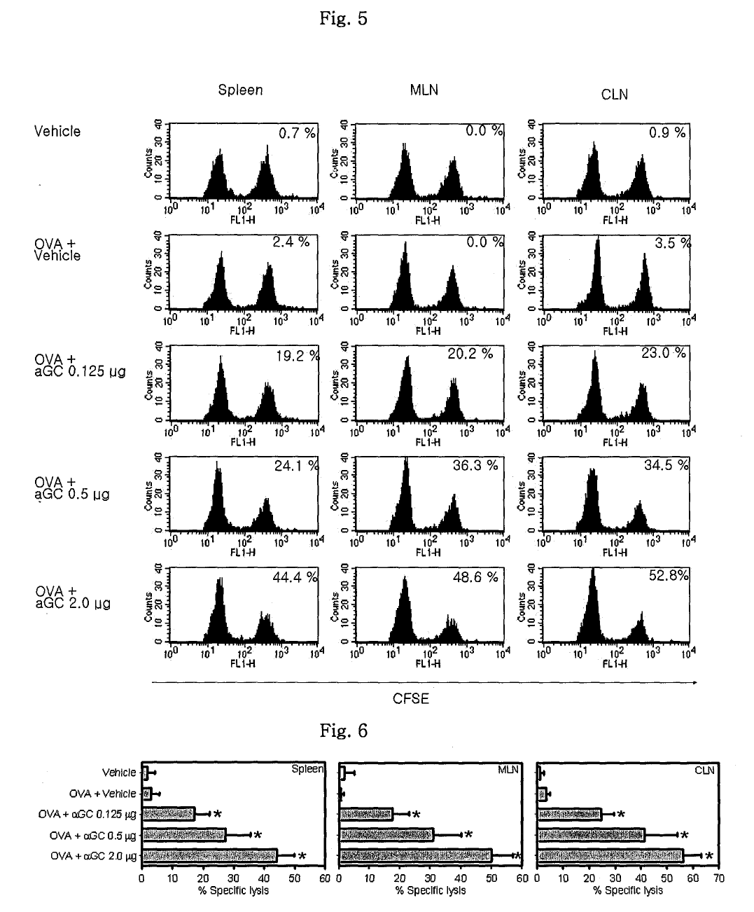

As shown in FIG. 5 and FIG. 6, all the groups coimmunized with α-GalCer nasal vaccine adjuvant showed higher cytotoxicity comparing with those with vehicle alone or OVA alone in a dose-dependent manner in spleen, MLN and CLN.

The above results indicate that αGalCer is a strong nasal vaccine adjuvant that is able to induce CTL in both local and systemic lymphatic organs.

### Example 4

### Humoral and Cell Mediated Immune Responses Induced by the Intranasal Co-Administration of an Antigen and αGalCer to Balb/c Mice

### <4-1> Measurement of an Antibody and a Cytokine (Humoral Immunity)

To investigate whether αGalCer can be used as a strong adjuvant for a nasal vaccine in Balb/c mice, different amounts of αGalCer (0.15, 0.5, 2.0 μg) and 100 μg of OVA were intranasally administered to Balb/c mice by the same manner as described in Example 1, followed by measurement of OVA-specific IgG, OVA-specific IgG1 and IgG2a in serum and OVA-specific IgA responses in the nasal wash and the lung wash.

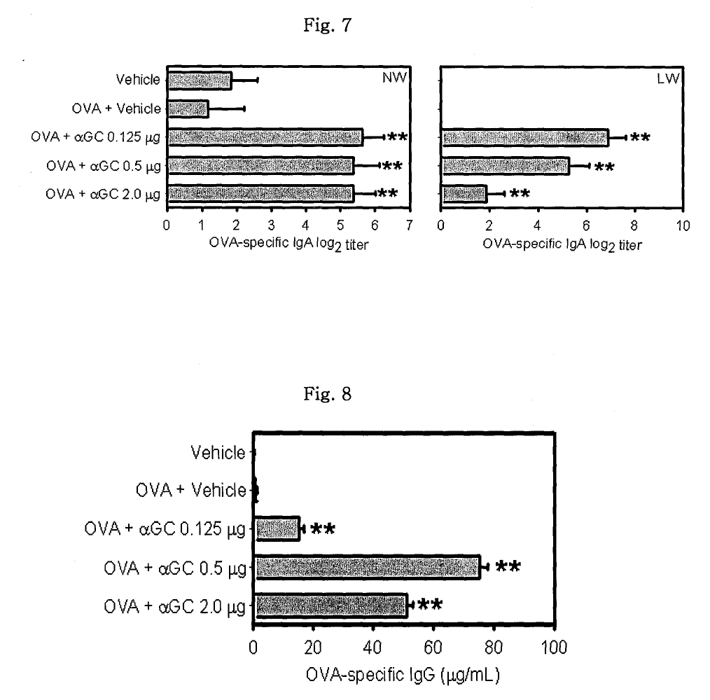

As shown in FIG. 7 and FIG. 8, the intranasal administration of αGalCer and OVA to Balb/c mice (Charles River Laboratories, Oriet Co., Ltd., Korea) induced higher OVA-specific IgG response in serum and higher OVA-specific IgA responses in the nasal wash and the lung wash, compared with those in mice treated with vehicle alone or OVA alone.

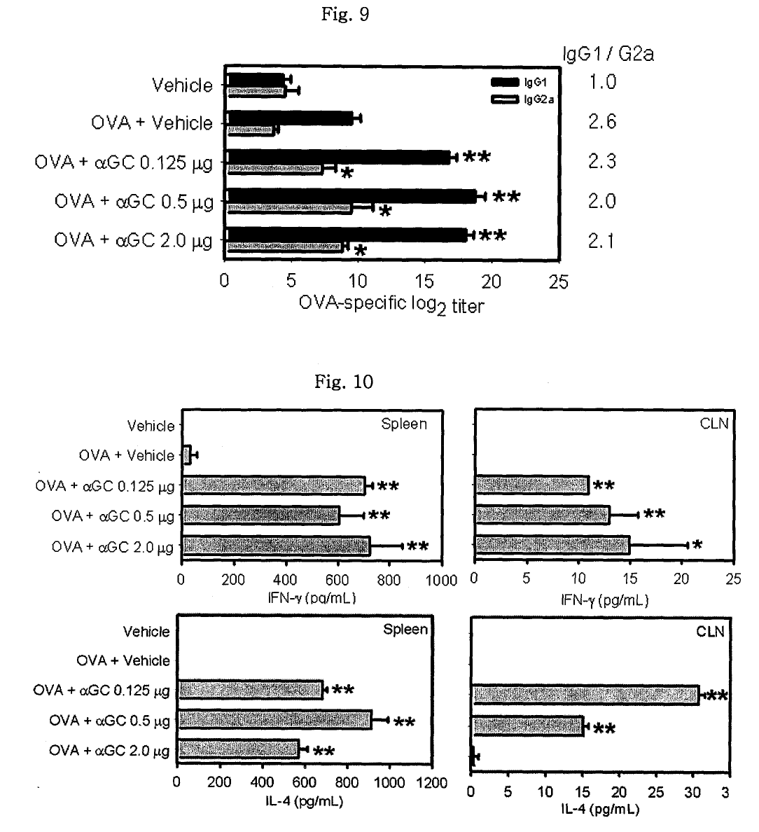

As shown in FIG. 9, the intranasal administration of αGalCer and OVA resulted in the increases in OVA-specific IgG1 and IgG2a titers.

As described in Example 2, different amounts of αGalCer (0.125, 0.5, 2.0 μg) and OVA were intranasally administered to Balb/c mice (Charles River Laboratories, Oriet Co., Ltd., Korea), followed by measurement of the levels of IFN-γ and IL-4 in spleen and CLN.

As shown in FIG. 10, all groups coimmunized with αGalCer showed significant increase in the production of IFN-γ and IL-4. Interestingly, when 0.5 μg of αGalCer was intranasally coadministered, the highest level of IgG antibody was detected in serum and the highest level of IL-4 was detected in spleen. Besides, the level of mucosal IgA in the lung wash and the production of IL-4 in CLN were in inverse proportion to the amount of αGalCer.

In conclusion, high concentration of αGalCer can induce tolerance against coadministered antigen in Balb/c mice.

### <4-2> Measurement of Cytotoxicity (Cell Mediated Immunity)

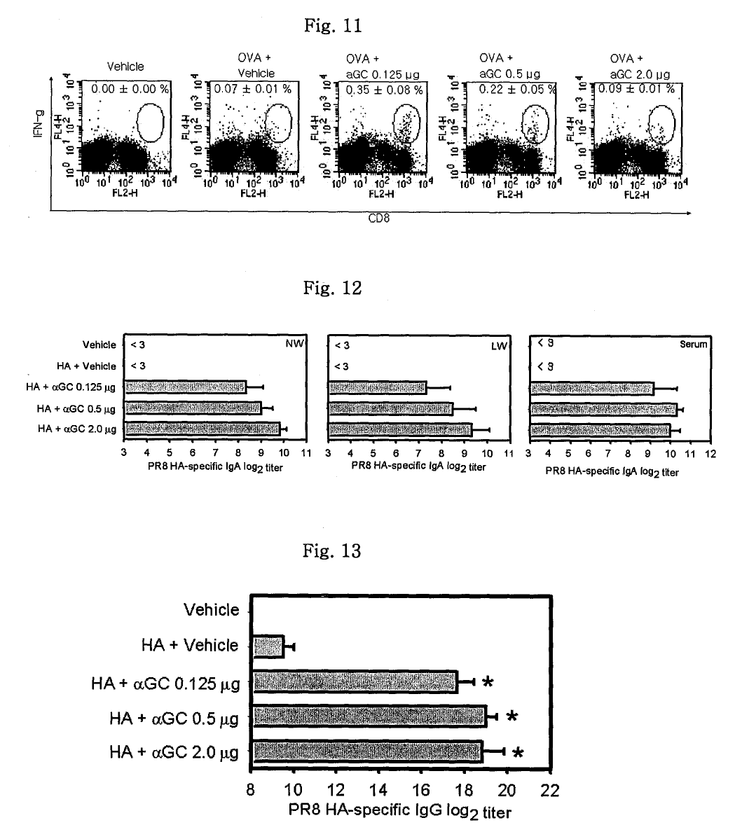

OVA dose not include an epitope peptide binding to a MHC class I molecule in Balb/c mouse. So, to investigate cytotoxic activity induced by αGalCer adjuvant in the Balb/c mouse, the numbers of IFN-γ-producing CD8+ T cells were measured (FIG. 11). Particularly, the cells (2×10⁶ cells/ml) were cultured for 4 days with 500 μg/ml of OVA, to which 1 μl/ml of GolgiPug™ (BD Pharmigen, USA) was added 6 hours before termination of the culture. Then, staining was performed by using FITC-conjugated CD3 mAb (Clone 145-2C11, Biolegend Inc, USA), PE-conjugated CD8 mAb (Clone 53-6.7, Biolegend Inc, USA) and APC-conjugated IFN-γ mAb (Clone XMG1.2, Biolegend Inc, USA). Intracellular staining was performed with BD Cytofix/Cytoperm Plus™ (BD Pharmigen, USA) according to the manufacturer's instruction, and the stained cells were analyzed with FACSCalibur (BD Bioscience, USA) and CellQuest software (BD Bioscience, USA).

As shown in FIG. 11, the numbers of IFN-γ-producing CD8+ T cells were decreased with the increase of αGalCer concentration. In FIG. 10, the amount of IFN-γ measured by sandwich ELISA did not depend on the concentration of αGalCer, but the numbers of IFN-γ-producing CTL were in inverse proportion to the concentration of αGalCer. The above results were attributed to the fact that the amount of IFN-γ detected by sandwich ELISA included all the IFN-γ secreted by different cells including CD4+, CD8+ T cells or APC but the numbers of CTL detected by FACS was only resulted from CD8+ T cells.

Therefore, the above results suggest that αGalCer has a strong nasal vaccine adjuvant activity in Balb/c mice.

### Example 5

### Anti-Virus Immune Response Induced by the Intranasal Co-Administration of αGalCer and a Virus Antigen Protein

To measure the degree of mucosal protection of αGalCer from virus infection, Balb/c mice were immunized with PR8 HA antigen (Dr. Shin-Ichi Tamura, Osaka University, Japan prepared by the method of Davenport, J. Lab. Clin. Med., 63:5, 1964) alone or together with αGalCer three times at one-week intervals. 2 weeks after the final immunization, the mice were infected with 20LD₅₀ of live influenza virus A/PR/8/34 through the nasal cavity. Three days after the virus infection, the nasal wash, the lung wash and serum were prepared and PR8 HA-specific antibody responses therein were measured by the same manner as described in Example 1. In addition, the weight loss and survival rate of the infected mice were observed every other day for 14 days.

As shown in FIG. 12, high levels of PR8 HA-specific S-IgA antibody were detected in the nasal wash and the lung wash and serum separated from all the groups coimmunized with αGalCer. As shown in FIG. 13, high level of PR8 HA-specific IgG antibody was also detected in the serum of the groups coimmunized with αGalCer.

The above results indicate that αGalCer can be used as a strong nasal vaccine adjuvant that is able to induce mucosal S-IgA antibody and systemic IgG antibody responses against a virus antigen.

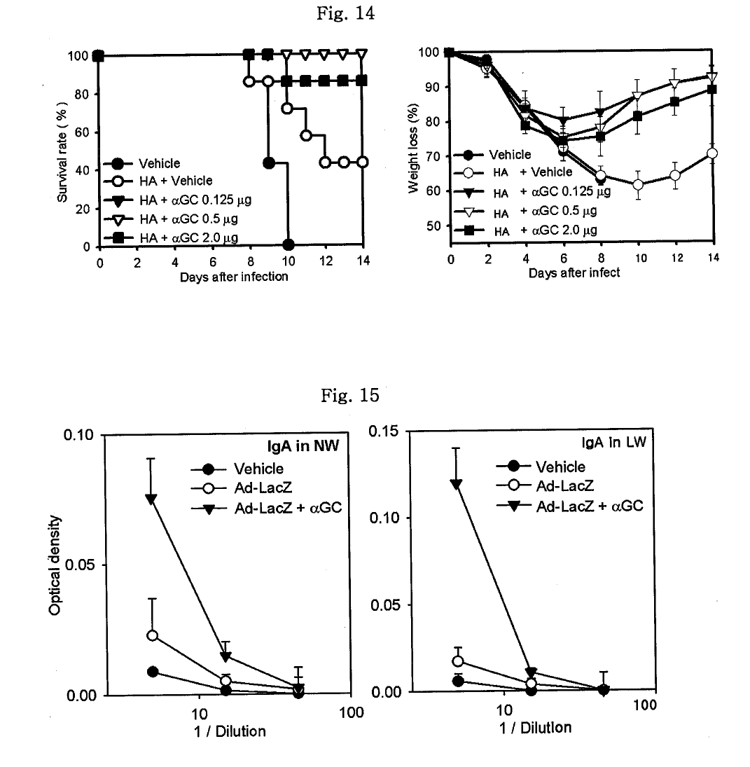

As shown in FIG. 14, more severe pathogenesis were observed in mice immunized without αGalCer, compared with those co-treated with an antigen and αGalCer, which was consistent with the results of measuring the survival rate, weight loss and weight recovery time. In the group treated with vehicle alone, all mice died within 10 days after the virus infection. In the group treated with PR8 HA alone, 57% of mice died within 14 days after the infection. However, the groups co-administered with PR8 HA and αGalCer through the nasal cavity didn't show any significant decrease in survival rate.

The above results indicate that αGalCer can be used as a strong nasal vaccine adjuvant that is able to induce mucosal S-IgA antibody and systemic IgG antibody responses, resulting in the protection against the virus infection.

### Example 6

### Anti-Virus Immune Response Induced by the Intranasal Co-Administration of αGalCer and Live Virus

Balb/c mice were immunized with 10⁶ pfu of replication-deficient live adenovirus harboring beta-galactosidase gene (Ad-LacZ) (Viromed, Korea) alone or together with 0.125 μg of αGalCer by the intranasal administration, two times at two-week intervals. One week after the final immunization, the nasal wash, the lung wash and serum were separated, by the same manner as described in Example 1, to measure β-galactosidase-specific antibody response. In addition, to measure CTL activity, spleen cells were stimulated by 2.5 μg/mL of β-galactosidase for 5 days and IFN-γ-producing CD8+ T cells were examined by intracellular cytokine staining according to the procedure as described in Example <4-2>.

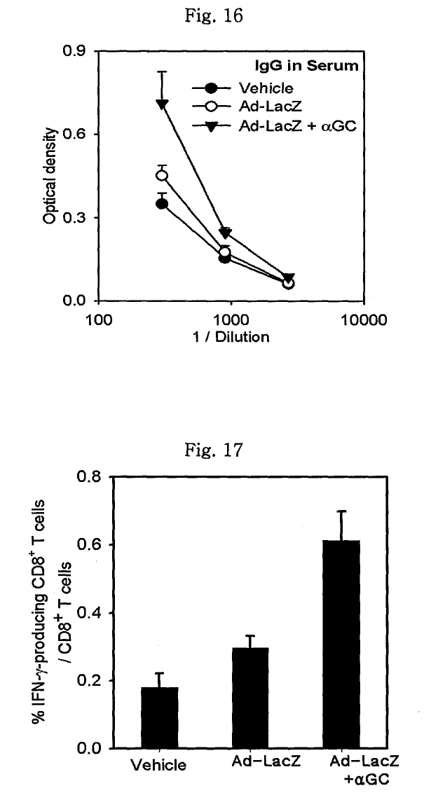

As shown in FIG. 15 and FIG. 16, higher levels of β-galactosidase-specific S-IgA antibody and β-galactosidase-specific IgG antibody were detected respectively in the nasal wash (NW) and the lung wash (LW) and in serum of the group coimmunized with Ad-LacZ and αGalCer by the concurrent intranasal administration than in those of the group immunized with vehicle alone or Ad-LacZ alone.

As shown in FIG. 17, significant increase in the numbers of IFN-γ-producing CD8+ T cells was confirmed in the group coimmunized with an antigen and αGalCer by the concurrent intranasal administration.

The above results indicate that αGalCer is an effective nasal vaccine adjuvant against the replication-deficient live virus.

### Example 7

### Anticancer Immune Response Against EG7 Tumor Induced by the Intranasal Co-Administration of an Antigen And αGalCer

To confirm whether αGalCer could be used as a nasal vaccine adjuvant inducing anticancer activity, C57BL/6 mice were immunized with 100 μg of OVA alone or together with αGalCer (0.125, 0.5, 2.0 μg) by the intranasal administration three times at one-week intervals. Two weeks after the final immunization, 3×10⁶ EG7 tumor cells were subcutaneously inoculated in the left flank of the immunized mice. On the 14^{th} day of the inoculation, the mice were sacrificed and the palpable tumors were weighed.

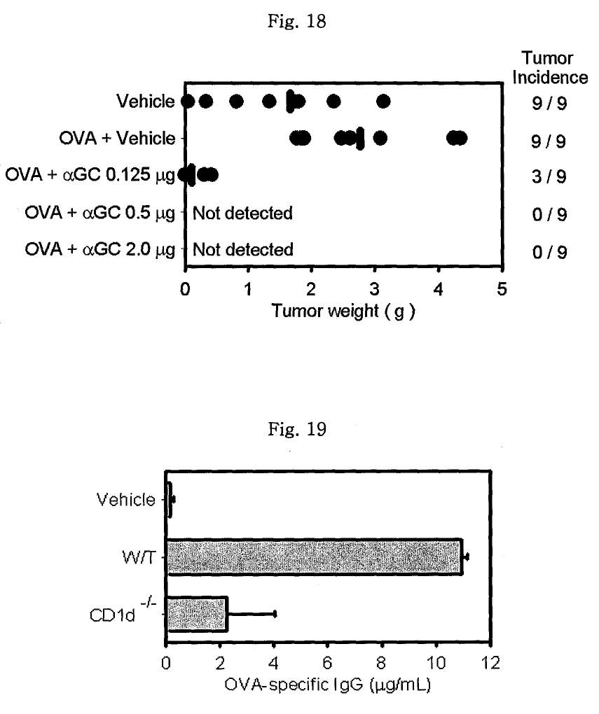

As shown in FIG. 18, tumor masses were found in all mice coimmunized with vehicle alone or OVA alone and in ⅓ of the mice treated with 0.125 μg of αGalCer. The tumors of the mice treated OVA alone through the nasal cavity were significantly heavy, compared with those of the mouse treated with vehicle alone (p<0.05). Interestingly, tumor formations were completely inhibited in mice treated with 0.5 μg and 2.0 μg of αGalCer together with OVA through the nasal cavity.

From the result, it was confirmed that αGalCer can be used as an effective and strong nasal vaccine adjuvant inducing anticancer immune response.

### Example 8

### CD1d Mediated Intranasal Adjuvant Activity of αGalCer

To investigate whether the immune responses induced by αGalCer were mediated by CD1d, NKT deficient (resulted from the lack of CD1d) CD1d−/− C57BL/6 mice (Charles River Lab., Orient Co. Ltd., Korea) were used for the experiment (Park et al., J. Exp. Med., 193: 893, 2001). On the first week of the final intranasal administration, systemic IgG level in serum and in vivo CTL activity were measured in both wild type and CD1d−/− C57BL/6 mice by the same manner as described in Example 1 and Example 3.

As shown in FIG. 19, systemic IgG antibody response was significantly inhibited in CD1d−/− mice.

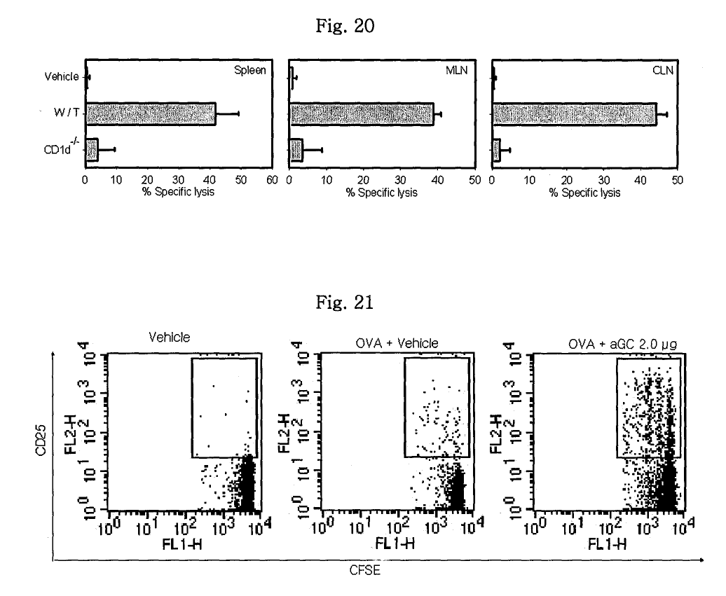

As shown in FIG. 20, CTL lytic activity was inhibited in draining lymph node and systemic lymphoid organs of CD1d−/− mice. The above results indicate that the immune responses induced by αGalCer of the invention were exclusively mediated by CD1d and KNT cells.

### Example 9

### Activation of Naïve T Cells and Differentiation Of the Activated T Cells into Effector Cells by the Intranasal Co-Administration of an Antigen and αGalCer

To investigate the effect of αGalCer on the activation of T cells, the surface expression of CD25 in CFSE-labeled OT1 cells (OVA specific CD8+ T cells), which were adoptively transferred into syngenic mice, was measured. OT1 cells were separated from OT1 mouse by using CD8α (Ly-2) magnetic bead (Mitenyl Biotech), which were labeled with 10 μM of CFSE at 37° C. for 15 minutes and then transferred intravenously into a syngenic mouse. One day after the adoptive transfer, the intranasal administration of 100 μg of OVA alone or together with 2.0 μg of αGalCer was performed thereto. 48 hours later, the expression of CD25 in CLN was investigated with FACS.

As shown in FIG. 21, the level of OT1 cells expressing CD25 was higher in the mice concurrently administered with OVA and αGalCer than those treated with OVA alone, indicating that αGalCer nasal adjuvant induces the activation of naïve T cells.

To confirm whether the activated T-cells were differentiated into fully functional CTL, 2×10⁶/ml of cells were further stimulated with 5 μM of OVA₂₅₇₋₂₆₄ peptide for 6 hours, by the same manner as described in Example 4, and then intracellular IL-2 and IFN-γ levels were measured by using APC-conjugated IL-2 (Clone JES6-5H4, Biolegend Inc., USA) and APC-conjugated IFN-γ mAb (Clone XMG1.2 Biolegend Inc., USA).

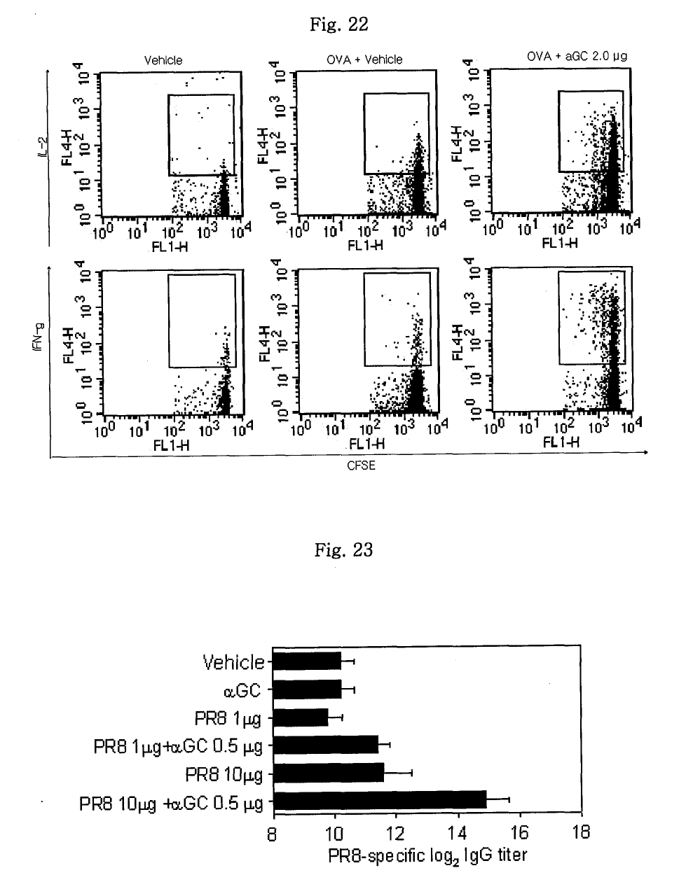

As shown in FIG. 22, the levels of OT1 cells secreting IL-2 and IFN-γ were higher in mice concurrently administered with OVA and αGalCer than those treated with OVA alone.

The above results indicate that the intranasal administration of αGalCer induces the activation of naïve T-cells and the differentiation of those activated T-cells into strong effector T cells.

### Example 10

### Anti-Virus Immune Response Induced by the Intranasal Co-Administration of αGalCer and a Killed Virus

To examine the role of αGalCer as an adjuvant of a killed virus, influenza virus A/PR/8/34 (PR8), which was inactivated with formalin, was used as an antigen to examine the anti-virus immune response. Balb/c mice were immunized with indicated amounts (1 μg, 10 μg) of inactivated PR8 alone or together with αGalCer by the intranasal administration twice at two-week intervals. Two weeks after the final immunization, the mice were sacrificed and following experiments were performed.

### <10-1> Investigation of Humoral Immune Response

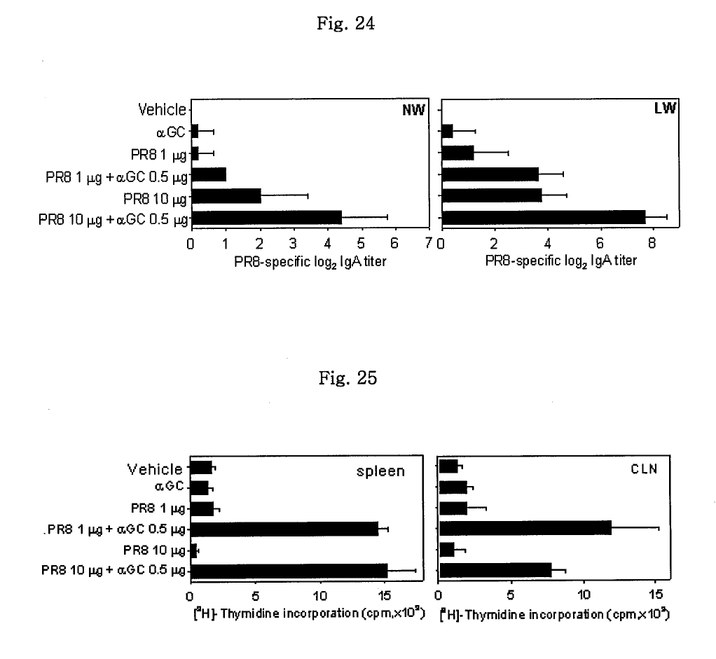

The nasal wash, the lung wash and serum were separated from the sacrificed mice and the antibody productions were observed therein by the same manner as described in Example 1. As shown in FIG. 23, comparison was made between the mice group treated with inactivated PR8 alone and that concurrently treated with the same amount of inactivated PR8 and αGalCer. As a result, the level of antigen-specific systemic IgG was significantly higher in mice concurrently administered with inactive PR8 and αGalCer than that treated with inactive PR8 alone. As shown in FIG. 24, the levels of mucosal S-IgA in the nasal wash and the lung wash were remarkably increased in the group concurrently administered with inactivated PR8 and αGalCer.

The above results confirmed that the concurrent intranasal immunization with αGalCer and a killed virus strongly induces potent humoral immune response.

### <10-2> Investigation of Immune Cell Proliferation

Single cells separated from the spleen and CLN of the sacrificed mice were cultured with inactivated PR8 for 3 days and [³H]-thymidine was added and further incubated for 18 hrs. As cells were being proliferated, the level of incorporated [³H]-thymidine was measured by LSC. As shown in FIG. 25, the proliferation of immune cells was significantly increased in mice concurrently administered with αGalCer.

The above result indicates that the intranasal immunization with αGalCer and a killed virus strongly induces the immune cell proliferation.

### <10-3> Productions of IFN-γ and IL-4 Induced by αGalCer

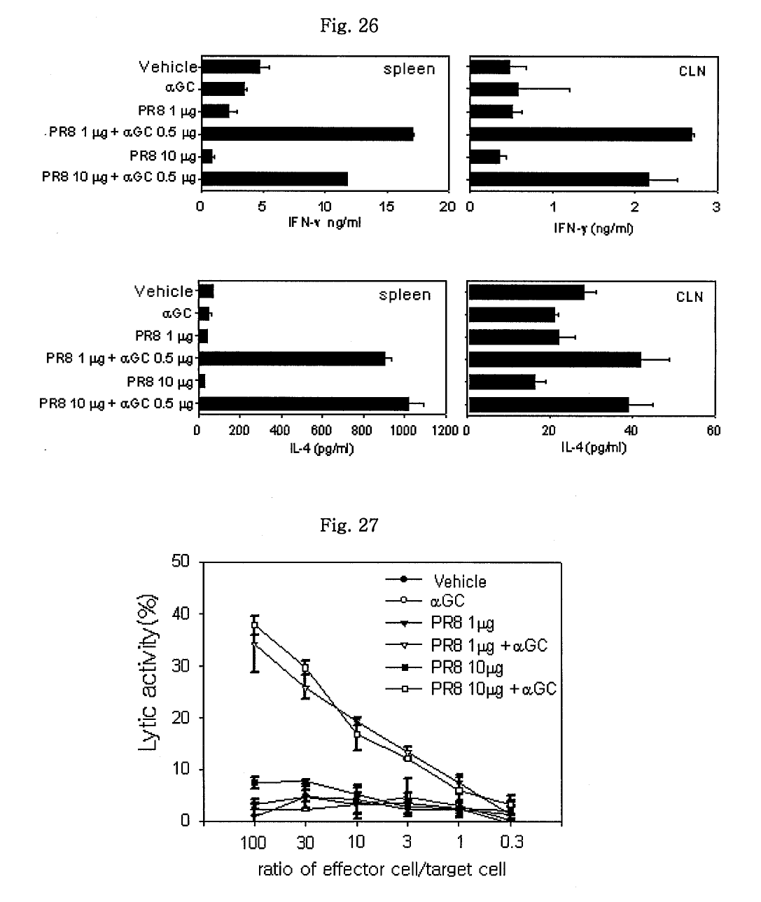

Single cells separated from the spleen and CLN of the sacrificed mice were cultured with inactive PR8 for 5 days. The supernatants were obtained and the levels of IFN-γ and IL-4 therein were measured by the same manner as described in Example 2. As shown in FIG. 26, the levels of Th1 cytokine IFN-γ and Th2 cytokine IL-4 were significantly increased in the spleen and CLN of the mice concurrently administered with αGalCer.

The above results indicate that the intranasal immunization with αGalCer and a killed virus induces Th1 and Th2 immune responses simultaneously.

### <10-4> Investigation of Cell Mediated Immune Response

Single cells, separated from the spleen of the sacrificed mice, were cultured with stimulator cells for 5 days. To obtain stimulator cells, single cells were taken from the spleen of a naïve Balb/c mouse, which was irradiated with γ-ray, resulting in the inactivation of the cells. Then, the inactivated cells were infected with a live PR8 virus. After culturing splenocytes with stimulator cell for five days, effector cells were three-fold diluted serially, followed by further culture with ⁵¹Cr-labeled target cells for 6 hours. Then, the amounts of ⁵¹Cr remaining in the culture supernatant were measured. The target cells were prepared by infecting P815 tumor cells (purchased from ATCC) with live PR8 virus and labeled with ⁵¹Cr. As shown in FIG. 27, target cell-specific lytic activity was observed only in mice concurrently treated with αGalCer.

The above result indicates that the concurrent intranasal immunization with αGalCer and a killed virus induces a strong cell mediated immune response.

### <10-5> Investigation of Protective Immune Response

As described hereinbefore, the concurrent intranasal immunization with αGalCer and a killed virus induced a strong humoral immune response and cell mediated immune response. Following experiments were performed to examine whether such immune responses could elicit the protective immune response when a live virus invaded.

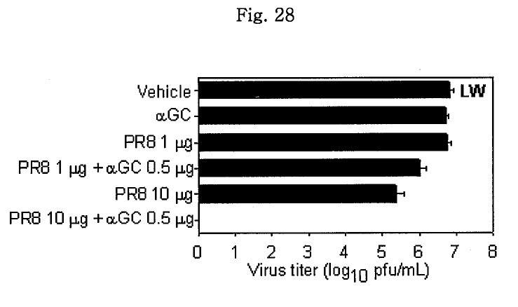

Immunized mice were infected with 20 LD₅₀ of live PR8 virus and sacrificed three days later to obtain the lung wash. The amounts of live PR8 virus in the lung wash were measured by plaque assay. Particularly, MDCK cells (purchased from ATCC) were cultured in a 6 well plate at the density of 95-100%. The lung wash was 10-fold diluted by using a medium serially, which was added to the plate, followed by infection for one hour. Then, the lung wash was eliminated. An agarose containing medium was added thereto, followed by further culture in a CO₂ incubator for 2-3 days. The numbers of plaques formed therein were counted with the naked eye. As shown in FIG. 28, no plaque was observed in mice concurrently immunized with 10 μg of inactivated PR8 and αGalCer, indicating that authentic protective immune response was induced.

The above results confirmed that the concurrent intranasal immunization with a killed virus and αGalCer induces a strong protective immune response.

### INDUSTRIAL APPLICABILITY

As explained hereinbefore, the present invention confirmed that the concurrent intranasal immunization with αGalCer and a tumor-associated antigen or a virus antigen effectively induces not only humoral immune response but also cell mediated immune response against the invaded tumor cells or a virus. Therefore, αGalCer of this invention can be effectively used as a nasal vaccine adjuvant for the prevention and treatment of virus infection and cancer.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A vaccine composition comprising an antigen and an effective dose of alpha-galactosylceramide (αGalCer) as an adjuvant for the intranasal administration.

2. The vaccine composition for the intranasal administration according to claim 1, wherein the antigen is selected from a group consisting of protein, recombinant protein, glycoprotein, peptide, polysaccharide, lipopolysaccharide and polynucleotide of a pathogen.

3. The vaccine composition for the intranasal administration according to claim 1, wherein the antigen is a cell or a virus.

4. The vaccine composition for the intranasal administration according to claim 1, wherein the alpha-galactosylceramide is included less than 0.5 w/v % as an adjuvant.

5. The vaccine composition for the intranasal administration according to claim 1, wherein the composition is formulated in the forms of liquid, powders or microspheres.

6. A method to enhance both systemic immune response and mucosal immune response against an injected antigen by the concurrent intranasal administration of the antigen together with alpha-galactosylceramide.

7. The method to enhance immune responses against an injected antigen according to claim 6, wherein the antigen and alpha-galactosylceramide are intranasally injected by the dispensing device.

8. The method to enhance immune responses according to claim 7, wherein the dispensing device is in the form of an aerosol or a drop delivery system.

9. A method to enhance both Th1 and Th2 immune responses by the intranasal administration of the vaccine composition of claim 1.

10. A method to enhance both IgA mucosal immune response and IgG systemic immune response by the intranasal administration of the vaccine composition of claim 1.

11. A vaccine adjuvant comprising alpha-galactosylceramide for the intranasal administration.
